(19) United States
(12) Patent Application Publication
(10) Pub. No.: US 2012/0220804 A1
(43) Pub. Date: Aug. 30, 2012

(54) MANUFACTURE OF DIMETHYL ETHER FROM CRUDE METHANOL

(75) Inventors: Peter Mitschke, Maintal (DE); Eckhard Seidel, Frankfurt am Main (DE); Thomas Renner, Frankfurt am Main (DE); Martin Rothaemel, Frankfurt am Main (DE)

(73) Assignee: LURGI GMBH, Frankfurt am Main (DE)

(21) Appl. No.: 13/509,832

(22) PCT Filed: Oct. 25, 2010
(86) PCT No.: PCT/EP10/06498

§ 371 Date: May 15, 2012

(30) Foreign Application Priority Data
Nov. 17, 2009 (DE) 10 2009 053 357.5
Nov. 17, 2009 (EP) 09014332.2

(51) Int. Cl. C07C 41/09; B01J 19/00; C07C 31/04

(52) U.S. Cl. 568/698; 568/840; 422/129

(57) ABSTRACT

A method of producing dimethyl ether by catalytic dehydration of crude methanol as feedstock in the gas phase includes providing the crude methanol from methanol synthesis, where the crude methanol having a total content of carbonyl compounds of not more than 100 wt-ppm, calculated, as mass equivalents of acetone. The crude methanol is evaporated, and the reaction temperature and reaction pressure are adjusted. A reactor filled with dehydration catalyst is charged with the evaporated crude methanol with a defined space velocity. A gaseous product mixture comprising dimethyl ether, non-reacted methanol and water is discharged. Cooling, partial condensation and separation of the gaseous product mixture are carried out so as to provide gaseous dimethyl ether, liquid water and methanol as products, and the methanol is recirculated.

This application is a U.S. National Phase application under 35 U.S.C. §371 of International Application No. PCT/EP2010/006498, filed on Oct. 25, 2010, and claims benefit to European Patent Application No. 09014332.2, filed on Nov. 17, 2009 and German Patent Application No. DE 10 2009 053 357.5, filed on November 17, 2009. The International Application was published in German on May 26, 2011 as WO 2011/060869 A1 under PCT Article 21 (2).

### FIELD

This invention relates to the production of dimethyl ether from crude methanol. In particular, this invention relates to a process for producing dimethyl ether by catalytic dehydration of crude methanol in the gas phase, and to a feedstock with the use of which a stable long-term operation of the process in accordance with the invention can be ensured. This invention furthermore relates to a plant for performing the process in accordance with the invention.

### BACKGROUND

The catalytic production of dimethyl ether (DME) from methanol by catalytic dehydration is known for many years. The U.S. Pat. No. 2,014,408 for example describes a process for the production and purification of DME from methanol on catalysts such as aluminum oxide, titanium oxide and barium oxide, with temperatures of 350 to 400° C. being preferred.

Further information on the conventional practices and on the current practice of the production of dimethyl ether can be found in Ullmann's Encyclopedia of Industrial Chemistry, Sixth Edition, 1998 Electronic Release, keyword “dimethyl ether”. In chapter 3 “Production” it is explained in particular that the catalytic conversion of pure, gaseous methanol is performed in a fixed-bed reactor, and after a two-stage condensation the reaction product then is supplied to a distillation, in which the DME product is separated from a methanol-water mixture. The methanol-water mixture then is separated in a second column, wherein the water is withdrawn from the process and the methanol is again recirculated into the DME reactor.

It should be emphasized that the current industrial practice consists in using pure methanol for producing DME, as it is explained by Vishwanathan et al., Applied Catalysis A: General 276 (2004) 251-255. Pure methanol here is understood to be a purified, largely anhydrous product of methanol synthesis. The direct product of methanol synthesis, on the other hand, is referred to as crude methanol and beside several wt-% of water also contains higher alcohols, ethers, esters, ketones, aldehydes, hydrocarbons and dissolved synthesis gas constituents each in trace amounts (Ullmann's Encyclopedia of Industrial Chemistry, Sixth Edition, 1998 Electronic Release, keyword “Methanol”, chapter 4.1.3 “Byproducts”).

The production of pure methanol from the direct product of methanol synthesis, the crude methanol, generally is effected by multistage distillation or rectification, wherein in the first step in a so-called low-boiler column the constituents with a lower boiling point than methanol are separated as top products; also with regard to the removal of dissolved gases, this intermediate product is referred to as stabilized crude methanol. Occasionally, there is also initially effected a distillative partial separation of water, wherein the methanol product obtained also is still referred to as crude methanol. Subsequently, largely anhydrous pure methanol is obtained as top product in at least one further distillation (Ullmann's Encyclopedia of Industrial Chemistry, Sixth Edition, 1998 Electronic Release, keyword “Methanol”, chapter 5.4 “Distillation of Crude Methanol”).

The production of pure methanol from crude methanol involves a great expenditure of both apparatus and energy, since in the methanol purification column large amounts of the low-boiling methanol must be separated from smaller amounts of the high-boiling water. For the use of pure methanol in the succeeding DME production, this represents an economic burden, since the methanol must again be evaporated completely. Therefore, the demand exists for quite some time to provide a practically useful process for producing DME proceeding from crude methanol. The unexamined German Patent Application DE 3817816 A1 for example describes a process integrated in a methanol synthesis for the catalytic production of DME from methanol by using dehydration catalysts, which is characterized in that the mixture emerging from the methanol synthesis reactor is at least partly reacted in a dehydration reactor on a suitable catalyst, preferably γ-Al2O3, for recovering DME, without previous separation of the non-reacted synthesis gas and without purification of the methanol produced.

The U.S. Pat. No. 6,740,783 B1 describes a process for producing DME from crude methanol. Here, it is explained that when using commonly used alumina-based dehydration catalysts, the activity of the catalyst is impaired by the water content in the crude methanol. As a solution it is proposed to use a hydrophobic zeolite as dehydration catalyst, which is less strongly deactivated in the presence of water. In addition, the binding of water to strongly Lewis acidic centers of the zeolite catalyst should suppress the carbonization of the catalyst.

A similar approach is made in the U.S. Patent Application US 2009/0023958 A1. Again, it is the object underlying the invention to provide a process for the catalytic dehydration of crude methanol in the gas phase. According to the inventors, this object is solved in that the crude methanol feed stream is passed first over a metal-doped, hydrophobic zeolite catalyst and subsequently over a catalyst selected from γ-Al2O3 or SiO2/Al2O3, wherein the dehydration reaction is performed in an adiabatic reactor. According to the inventors, this combination of process features should have advantages with respect to the temperature guidance in the reactor, the low formation of byproducts and the lower catalyst deactivation.

Altogether, it should therefore be noted that in various processes or process variants for producing dimethyl ether by catalytic dehydration of crude methanol in the gas phase have already been proposed, but the proposed processes have not gained acceptance in the industrial practice. Despite the relevant prior art discussed above, all technical plants for producing dimethyl ether by catalytic dehydration of methanol in the gas phase today still operate by using pure methanol as feedstock. Despite the described economic advantages, fundamental disadvantages therefore seem to exist when using crude methanol as feedstock, which could not be overcome to this date.

### SUMMARY

In an embodiment, the present invention provides method of producing dimethyl ether by catalytic dehydration of crude methanol as feedstock in the gas phase that includes providing the crude methanol from methanol synthesis, where the crude methanol having a total content of carbonyl compounds of not more than 100 wt-ppm, calculated as mass equivalents of acetone. The crude methanol is evaporated and the reaction temperature and reaction pressure are adjusted. A reactor filled with dehydration catalyst is charged with the evaporated crude methanol with a defined space velocity. A gaseous product mixture comprising dimethyl ether, non-reacted methanol and water is discharged. Cooling, partial condensation and separation of the gaseous product mixture are carried out so as to provide gaseous dimethyl ether, liquid water and methanol as products, and the methanol product is recirculated.

### DETAILED DESCRIPTION

An aspect of the present invention provides a process for producing dimethyl ether by catalytic dehydration of crude methanol in the gas phase, which avoids the above-mentioned disadvantages and which is suitable for industrial use.

In an embodiment, the present invention provides by a process for producing dimethyl ether by catalytic dehydration of crude methanol in the gas phase, which comprises the following process steps:

(a) providing crude methanol from the methanol synthesis,

(b) evaporating the crude methanol, possibly after previous stabilization and/or after partial separation of water and adjusting a reaction temperature and a reaction pressure,

(c) charging a reactor filled with dehydration catalyst with the evaporated crude methanol with a defined space velocity,

(d) discharging a gaseous product mixture, comprising dimethyl ether, non-reacted methanol and water,

(e) cooling, partial condensation and separation of the gaseous product mixture, wherein gaseous dimethyl ether as well as liquid water and methanol are obtained as products, wherein the methanol is recirculated to process step 1 (a), and which is characterized in that the crude methanol used as feedstock has a total content of carbonyl compounds of not more than 100 wt-ppm, preferably not more than 50 wt-ppm, calculated as mass equivalents of acetone.

It was found that in the production of dimethyl ether by catalytic dehydration of crude methanol in the gas phase the content of carbonyl compounds in the crude methanol has a decisive importance for the long-term stability of the process. This is surprising, since the negative effects of oxygen-containing trace components on the performance of the production process or the plant used for this purpose in the production of DME proceeding from crude methanol so far have not been discussed or even denied in the prior art. The International Patent Application WO 01/21561 A1 for example teaches that in the production of short-chain olefins from methanol, which takes place via the intermediate product DME, the presence of organic, oxygen-containing trace components such as higher alcohols, aldehydes or other oxygenated compounds only has an insignificant influence on the reaction. By contrast, it has now been found that when exceeding a limit value of 100 wt-ppm for the total content of carbonyl compounds in the crude methanol feedstock, calculated as mass equivalents of acetone, a multitude of additional trace components appear in the DME product, which are undesirable as impurities. This applies in particular for the case that only the acetone is contained in the crude methanol as carbonyl compound. However, when the crude methanol feedstock also contains higher, potentially more reactive carbonyl compounds such as methyl ethyl ketone (MEK), a total content of carbonyl compounds in the crude methanol of not more than 50 wt-ppm is preferred, since it has been observed that when maintaining this limit value no unknown, potentially harmful trace components appear in the DME product.

It has also been found that due to condensation or polymerization reactions these trace components form solid products which lead to the formation of deposits inside the plant and/or on the catalyst, which results in the clogging of plant sections such as heat exchangers or the premature deactivation of the catalyst. Such deposits have been observed in corresponding experiments described below. As an important ingredient of the deposits hexamethylbenzene (HMB) could be identified by means of an analytical determination. The same is obtained in a manner known per se from the reaction of methanol with acetone and due to its high melting point of 165° C. leads to the formation of solid deposits in colder plant sections and to the carbonization of the catalyst. This reaction is described by Jayamani et al, Indian Journal of Chemistry, Section B: Organic Chemistry Including Medicinal Chemistry (1985), 24B(6), 687-9, for the preparative production of HMB. In the Journal of Catalysis, 119, 288-299 (1989), Ganesan and Pillai also describe the reaction of methanol with different ketones and aldehydes on an Al2O3 catalyst to obtain hexamethylbenzene (HMB), wherein at 350° C. acetone and MEK are converted for 100% and HMB is obtained with yields of 87 to 90%. Seen mechanistically, the reaction should always proceed via acetone—independent of the type of carbonyl compound, so that acetone appears to be an expedient reference component for indicating the total content of carbonyl compounds. This is of particular interest, since crude methanol contains these compounds and Al2O3 likewise is used as catalyst in the DME production by gas phase processes. Consequently, the undesired condensation reactions to obtain high-boiling compounds such as HMB can take place not only with the participation of acetone, but also in the presence of other carbonyl compounds. It should be considered, however, that in the experiments described in the paper of Ganesan and Pillai always very high concentrations of the carbonyl compounds of about 16 mol-% were used, which lies distinctly above the usual concentrations of these compounds in the crude methanol, which only amount to some ten to some hundred ppm.

Surprisingly, it was found that limit values for tolerable amounts of carbonyl compounds in the crude methanol can be defined, with the maintenance of which a stable long-term operation of the DME production plant is possible and no impurities are detected in the DME product in disturbing concentrations. It has been found that for a total content of carbonyl compounds of not more than 100 wt-ppm, calculated as mass equivalent of acetone, the side reactions proceed to such a subordinate extent that the plant operation and the catalyst are not negatively influenced. This applies in particular for the case that only the acetone is contained in the crude methanol. However, when the crude methanol feedstock also contains higher, potentially more reactive carbonyl compounds such as methyl ethyl ketone (MEK), a total content of carbonyl compounds in the crude methanol of not more than 50 wt-ppm, calculated as mass equivalent of acetone, is preferred, since it has been observed that when maintaining this limit value no unknown, potentially harmful trace components appear in the DME product. Accordingly, corresponding limit values can be specified for a crude methanol determined as feedstock for the DME production, with the maintenance of which an undisturbed operation of the plant is still possible, and a sufficiently pure DME product is obtained.

In the production of dimethyl ether by catalytic dehydration of pure methanol in the gas phase, said effect does not occur, since the total content of carbonyl compounds in the pure methanol is very low, wherein usually only the acetone content is indicated. For example, pure methanol of the purity level “Grade AA” has an acetone content below 20 wt-ppm (Supp, E., How to Produce Methanol from Coal, Springer Verlag, Berlin (1989), p. 134). A more recent reference specification of the International Methanol Producers and Consumers Association states an acetone limit value of 30 mg/kg (January 2008, http://www.impca.be/).

It is assumed that the problem of the presence of oxygen-containing, organic trace components has not been discussed sufficiently in earlier papers on the catalytic dehydration of crude methanol in the gas phase to obtain DME, since in these papers the attention was directed to the water content of the crude methanol. In many of the examinations described in the prior art, synthetic crude methanol mixed together from the pure chemicals methanol and water possibly has been used instead of crude methanol originating from a technical plant for methanol synthesis, so that the above-mentioned problem could not be seen.

The U.S. Pat. No. 4,560,807 mentions the possibility of using, beside pure methanol, also a non-specified byproduct methanol with a higher content of other oxygenates as raw material for the DME production. In this connection, the compounds methyl ethyl ether, methyl formate and formal (dimethoxymethane) are mentioned. However, the indications merely relate to accumulations to be expected of these impurities in the DME product and not to their possibly harmful effects on the performance of the production process or on the plant itself which is used for this purpose. In the numerical example contained in the patent specification only pure methanol again is used.

In an embodiment of the invention, a fixed-bed reactor is used as reactor. This type of reactor is characterized by its constructive simplicity and has proven quite successful in the production of DME proceeding from pure methanol.

An advantageous aspect of the process of the invention provides to use γ-Al2O3 as catalyst. Other acidic solid catalysts can also be employed in the process of the invention, but γ-Al2O3 has some advantages with respect to its handling, its low toxicity as well as economic advantages.

In the process of the invention, the reaction temperature preferably lies between 200 and 500° C., particularly preferably between 250 and 450° C. The reaction pressure preferably lies between 1 and 100 bar(a), particularly preferably between 1 and 30 bar(a). Suitable space velocities were found to be values between 1 and 8 kg/(kg·h), preferably between 1 and 6 kg/(kg·h). The space velocity is defined as kg of methanol per h and per kg of catalyst.

Advantageously, stabilized crude methanol is used as feedstock for the process in accordance with the invention. The reduction of the content of dissolved gases in a stabilization column leads to a more stable plant operation in the catalytic dehydration of methanol in the gas phase, since outgassing is avoided in the crude methanol conduits or intermediate containers. In addition, potentially harmful gas constituents are kept away from the dehydration catalyst. Already with a low content of dissolved gases in the crude methanol, however, it can be advantageous to use crude methanol as feedstock without previous stabilization. The omission of the stabilization column leads to significant savings as regards the investment costs for the DME production plant.

In accordance with a preferred aspect of the invention, the product mixture obtained in process step 1 (e), comprising dimethyl ether, water and non-reacted methanol, is separated by means of distillation. Usual and commonly known techniques of distillation, fractional distillation or rectification can be employed. The dimethyl ether obtained after separation can subsequently be used as feedstock for the production of short-chain olefins, as fuel and/or propellant or as aerosol propellant gas in spray cans.

This invention also relates to a crude methanol suitable as feedstock for the production of dimethyl ether by catalytic dehydration in the gas phase, which is characterized in that it has a total content of carbonyl compounds of not more than 100 wt-ppm, preferably not more than 50 wt-ppm. If no further information is available on the type of ketones present, but only on the total content of carbonyl compounds as sum parameter, it is safer to maintain the lower limit value for the total content of carbonyl compounds of not more than 50 wt-ppm. If it is ensured, on the other hand, that only acetone is present as carbonyl compound in detectable concentrations, the higher limit value for the total content of carbonyl compounds of not more than 100 wt-ppm can be employed.

This invention furthermore relates to a plant for performing the process in accordance with the invention. It comprises means for performing the process steps according to claim 1 (a) to (e), in particular conduits and/or recipient tanks for providing crude methanol from the methanol synthesis, heat exchangers and/or heaters for evaporating the crude methanol and for adjusting a reaction temperature, means for adjusting the reaction pressure, a conveying means for the crude methanol, a reactor filled with dehydration catalyst, conduits for discharging the gaseous product mixture, heat exchangers and/or coolers for cooling the product mixture, a separating device for separating the product mixture, and conduits for recirculating the non-reacted methanol before the dehydration reactor. The plant is characterized in that it is operated with crude methanol as feedstock according to claim 2.

Further developments, advantages and possible applications of the invention can also be taken from the following description of embodiments and numerical examples. All features described form the invention per se or in any combination, independent of their inclusion in the claims or their back-reference.

### EMBODIMENT

Crude methanol is produced in a plant for the catalytic methanol synthesis by the low-pressure process and supplied to a stabilization column. In the stabilization column, the distillative separation of the crude methanol is effected, wherein the components with boiling points below that of the methanol are separated as top product. The stabilized crude methanol obtained as bottom product is supplied to an intermediate container. The water content of the stabilized crude methanol is 12 wt-%, its total content of carbonyl compounds is about 50 wt-ppm, calculated as acetone, and the acetone content is about 30 wt-ppm. The crude methanol is withdrawn from the intermediate container by means of a pump and is preheated or partly evaporated by means of a heat exchanger by indirect heat exchange against the hot product gases of the dehydration reactor. The final evaporation and the adjustment of the reaction temperature is effected in a downstream heat exchanger by direct heat exchange against high-pressure steam. The adjustment of the reaction pressure is effected by means of a pressure-maintaining valve on the exit side of the dehydration reactor. The DME reactor filled with lumpy γ-Al2O3 catalyst is charged with the crude methanol vapor brought to the reactor inlet temperature of 300° C. The methanol space velocity is 2.0 kg/(kg·h), the reaction pressure is 16 bar(a). Because of the comparatively low heat of reaction of the dehydration reaction, the DME reactor is configured as an adiabatic fixed-bed reactor. In the dehydration reactor, a partial conversion of the crude methanol to DME and water is effected corresponding to the equilibrium of the dehydration reaction in dependence on the temperature and the partial pressures of methanol and water. Under these conditions, the methanol conversion achieved lies between 75 and 82 wt-%; based on methanol used, the DME selectivity lies between 98 and 100 mol-C %.

The product gas is discharged from the dehydration reactor and cooled in a heat exchanger by indirect heat exchange with the colder crude methanol withdrawn from the intermediate container. The further cooling of the product gas is effected in a further water-cooled heat exchanger, wherein partial condensation of the water and of the non-reacted methanol occurs. The further processing of the product is effected in a manner known per se (Ullmann's Encyclopedia of Industrial Chemistry, Sixth Edition, 1998 Electronic Release, keyword “Dimethyl Ether”, chapter 3 “Production”) by two-stage distillation, wherein DME is obtained as top product in the first distillation stage. The DME obtained is liquefied in a downstream condenser and thus separated from low boilers, e.g. trace gas constituents. In this way, DME product purities of >99.9 % are achieved. In a downstream scrubber, the gaseous top product of the condenser is liberated from DME traces still present by using crude methanol as washing agent. The DME-laden crude methanol is recirculated to the dehydration reactor as feedstock. In the second distillation stage, methanol is obtained as top product, which likewise is recirculated to the dehydration reactor as feedstock. The waste water obtained as bottom product is removed from the process.

### NUMERICAL EXAMPLES

To elucidate the limit value for the total content of carbonyl compounds for a safer plant operation in the catalytic dehydration of crude methanol in the gas phase, a plurality of experiments were performed in a pilot plant with different acetone concentrations. The pilot plant consisted of a crude methanol supply, an evaporator and a final heater, a fixed-bed reactor of stainless steel with an inside diameter of 27.3 mm and a two-stage cooling and separation. The separation consisted of a gas/liquid phase separator, as whose products a condensate and a product gas were obtained. Analysis samples were taken from the crude methanol feedstock, from the condensate and from the product gas, wherein the product gas additionally was passed through a wash bottle filled with methanol, so as to be able to more accurately detect oxygen-containing trace constituents in the product gas. There was used a gas-chromatographic standard analysis method for crude methanol, by means of which alcohols, ethers, esters, ketones and hydrocarbons can be detected.

For all experiments, the following general experimental conditions were used:

Catalyst weight: 150 g

Type of catalyst: γ-Al2O3 as tablets (manufacturer: Süd-Chemie)

Reactor inlet temperature: 300° C.

Reactor pressure: 16 bar(a)

Space velocity: 2 kg/(kg·h) (as defined above)

### Examples 1 to 4 and Comparative Example 1

Experiments were performed with different acetone concentrations in the methanol feedstock with otherwise identical reaction conditions (Examples 1 to 4), wherein an experiment without addition of acetone was used as reference (Comparative Example 1). The essential results are listed in the following Table:

| | Comp. | | | | |
|---|---|---|---|---|---|
| | Example 1 | Example 1 | Example 2 | Example 3 | Example 4 |
| Water content in feedstock, wt-% | 12 | 12 | 12 | 12 | 12 |
| Acetone in feedstock, wt-ppm | 0 | 100 | 2,000 | 10,000 | 100,000 |
| Methanol conversion | 76% | 78% | 76-77% | 76% | n.d.* |
| DME yield, based on mol C | 99.9% | 99% | 98-99% | 98% | n.d.* |
| Unknown components (GC peaks) in | none | none | about 70 | about 160 | n.d.* |
| condensate | | | | | |
| Unknown components (GC peaks) in | none | none | about 90 | about 200 | n.d.* |
| product gas after absorption in | | | | | |
| methanol | | | | | |
| Clogging after runtime of | none | none | none | 1 day | <5 h |
| (maximum duration of 50 h) | | | | | |
| Ingredients of the solids | — | — | — | HMB | HMB |

*n.d. = not determined, due to the quick failure of the plant, a complete mass balance and analytics of the various product streams could not be performed.

It was found that at concentrations ≦100 wt-ppm of acetone in the feedstock no impairments of the conversion of methanol were observed (Example 1 as compared to Comparative Example 1). At concentrations of 2000 wt-ppm and more, a very large number of unknown products is formed, which are detected in the condensate and product gas (Example 2), but after the maximum operating period of 50 h no clogging was yet observed in the pilot plant. When the acetone concentration was increased to 10000 wt-ppm, the number of unknown reaction products increased distinctly, and after about 1 day of trial operation clogging was detected, so that the plant had to be shut down (Example 3). An analysis of the solids causing such clogging revealed that the same substantially consist of hexamethylbenzene (HMB). At an even higher acetone concentration of 100000 wt-ppm (10 wt-%, according to the above-discussed papers on the production of HMB) a regular trial operation could not be maintained, since the plant was clogged within less than 5 h of trial operation. Again, the deposits consisted of HMB.

### Example 5

In a further experiment, the influence of the MEK concentration was examined, which according to the prior art should behave similar to acetone and undergo similar reactions. At the conditions described above, the experiment was performed analogous to Examples 1 to 4. The results are listed in the following Table:

| | |
|---|---|
| Water content in feedstock, wt-% | 12 |
| Acetone in feedstock, wt-ppm | 0 |
| MEK in feedstock, wt-ppm | 2000 |
| Total content of carbonyl compounds | 1620 |
| (based on mass equivalents of acetone) ^{#)} | |
| Methanol conversion | 76% |
| DME yield, based on mol C | 98.2-99.6% |
| Unknown components (GC peaks) | about 100 |
| in condensate | |
| Unknown components (GC peaks) | about 100 |
| in product gas after absorption in | |
| methanol | |
| Clogging after runtime of | none |
| (maximum duration 430 h) | |
| Composition of the solids | — |

^{#)} calculated via the relationship: mass equivalents of acetone = wt-ppm of carbonyl compound × molar mass of acetone/molar mass of carbonyl compound

No clogging of the plant occurred, but it is also found here that many new unknown components are formed by side reactions of MEK and methanol. There even is a trend towards the formation of still more unknown components than at a comparable acetone concentration in the crude methanol feedstock (cf. Example 2); this can be substantiated in that MEK in contrast to acetone represents an unsymmetrically substituted ketone (one methyl and ethyl group each), whereby more combination possibilities exist for the formation of new products.

### Example 6

In a further experiment in the plant under identical conditions the influence of other impurities usually present in the crude methanol on the plant operation was determined. The results are listed in the following Table. The maximum duration of the experiment with this feed mixture was 430 h. In contrast to the previous experiments, the temperature was varied as well.

| | |
|---|---|
| Water content in feedstock, wt-% | 12 |
| Acetone in feedstock, wt-ppm | 0 |
| MEK in feedstock, wt-ppm | 60 |
| Total content of carbonyl compounds | 48 |
| (based on mass equivalents of acetone) | |
| Ethanol in feedstock, wt-ppm | 1000 |
| i-Propanol in feedstock, wt-ppm | 280 |
| sec-Butanol in feedstock, wt-ppm | 280 |
| Hexane in feedstock, wt-ppm | 200 |
| Reactor inlet temperature | 280-400° C. |
| Methanol conversion | 70-77% |
| DME yield, based on mol C | 98.7-99.7% |
| Unknown components (GC peaks) | 0 |
| in condensate | |
| Unknown components (GC peaks) | 0 |
| in product gas after absorption in | |
| methanol | |
| Clogging after runtime of | none |
| (maximum duration 430 h) | |
| Composition of the solids | — |

It can be seen that the presence of other oxygen-containing compounds, which occur in the crude methanol as impurities, has no negative effect on the dehydration of crude methanol, in case the required limit value of 50 wt-ppm is maintained for the total content of carbonyl compounds. This finding also applies for the distinctly higher temperatures examined.

### Example 7

To more accurately examine the effect of the undesired reaction of acetone with methanol to obtain HMB and other components, 64 g of methanol and 6.4 g of acetone were heated in an autoclave together with 173 g of γ-Al₂O₃ for 20 h at 230° C. and a pressure of 20 bar. After a duration of 20 h, the experiment was terminated and the catalyst was removed and analysed. Severe brownish discolorations could clearly be seen. Analyses of the catalyst in addition revealed changes of the BET surface and of the pore volume before and after the reaction, wherein before determination of BET surface and pore volume the used catalyst from Example 7 was annealed in inert gas at 500° C., in order to desorb low-volatility organic components. The experimental results are listed in the following Table.

| | | Used catalyst from |
|---|---|---|
| | | Example 7, |
| | Fresh catalyst | after annealing |
| BET surface, m²/g | 210 | 187 |
| Pore volume, m³/g | 0.480 | 0.378 |
| Weight loss due to | — | 18.3 wt-% |
| outgassing at 500° C. | | |

It can clearly be seen that due to the undesired side reactions, which take place at too large concentrations of acetone in the crude methanol, the BET surface and the pore volume decreased distinctly. When the 18.3 wt-% of adsorbed organic molecules are included in the calculation, the free pore volume decreases even further, e.g. with an assumed density of 1.5 g/cm³ for the adsorbates by about 0.12 m³/g to only about 0.26 m³/g as compared with 0.480 cm³/g for the fresh catalyst. Since the catalyst used is a bulk catalyst, other factors such as metal loading or metal dispersion are not relevant for the deactivation, but instead the catalytic activity primarily is determined by the physical accessibility of the catalytically active inner surface. Thus, due to the observed reduction of the BET surface and the pore volume it is to be expected that runtime and performance are reduced as compared to a proper operation, i.e. with a feedstock with a lower acetone concentration.

Thus, the presence of too high concentrations of carbonyl compounds not only leads to an impairment of the process due to the formation of deposits e.g. in pipe conduits, which each would lead to an undesired standstill of the plant and reduce the plant availability, but they also lead to a degradation of the catalyst and thus effect lower methanol conversions and DME yields.

### INDUSTRIAL APPLICABILITY

With the invention, an improved process for producing dimethyl ether thus is provided, which due to the use of crude methanol for dehydration is characterized by economic advantages as compared to a process based on pure methanol. In this way, at least one distillation stage is saved for the processing of crude methanol. Avoiding the distillation of large amounts of methanol as low boilers in the pure-methanol column significantly reduces the energy consumption of the process. The use of crude methanol for dehydration is unproblematic when the limit values indicated in the claims for the total content of carbonyl compounds are maintained. There is obtained a DME product which despite the use of crude methanol has a particularly low content of disturbing impurities.

While the invention has been particularly shown and described with reference to preferred embodiments thereof, it will be understood by those skilled in the art that various changes in form and details may be made therein without departing from the spirit and scope of the invention.

1-14. (canceled)

15. A method of producing dimethyl ether by catalytic dehydration of crude methanol as feedstock in the gas phase, the method comprising:
(a) providing crude methanol from, methanol synthesis, the crude methanol having a total content of carbonyl compounds of riot more than 100 wt-ppm, calculated as mass equivalents of acetone,
(b) evaporating the crude methanol and adjusting a reaction temperature and a reaction pressure,
(c) charging a reactor filled with dehydration catalyst with the evaporated crude methanol with a defined space velocity,
(d) discharging a gaseous product mixture comprising dimethyl ether, non-reacted methanol and water, and
(e) cooling, partially condensing and separating the gaseous product mixture so as to provide gaseous dimethyl ether, liquid water and methanol as products, and recirculating the methanol product to step (a).

16. The method recited in claim 15 wherein the crude methanol has a total content of carbonyl compounds of not more than 50 wt-ppm, calculated as mass equivalents of acetone.

17. The method recited in claim 15, wherein the reactor is a fixed-bed reactor.

18. The method recited in claim 15 wherein the catalyst is γ-Al₂O₃.

19. The method recited in claim 15 wherein a reaction temperature is between 200 and 500° C.

20. The method recited in claim 19 wherein the reaction temperature is between 250 and 450° C.

21. The method recited in claim 15 wherein a reaction pressure is between 1 and 100 bar(a).

22. The method recited in claim 21, wherein the reaction pressure is between 1 and 30 bar(a).

23. The method recited in claim 15, wherein the space velocity is between 1 and 8 kg/(kg·h).

24. The method recited in claim 23, wherein the space velocity is between 1 and 6 kg/(kg·h).

25. The method recited in claim 15 wherein the crude methanol is stabilized crude methanol.

26. The method recited in claim 15 wherein crude methanol is provided without previous stabilization.

27. The method recited in claim 15 wherein the separation of the gaseous product mixture includes distillation.

28. The method recited in claim 15, further comprising providing the produced dimethyl ether as feedstock for producing short-chain olefins.

29. The method recited in claim 15, further comprising providing the produced dimethyl ether as fuel to a subsequent process.

30. The method recited in claim 15, further comprising providing the produced dimethyl ether as aerosol propellant gas to a subsequent process.

31. Crude methanol as feedstock for producing dimethyl ether by catalytic dehydration of crude methanol in the gas phase, wherein the crude methanol has a total content of carbonyl compounds of not more than 100 wt-ppm, calculated as mass equivalents of acetone.

32. The crude methanol of claim 31 wherein the crude methanol has a total content of carbonyl compounds of not more than 50 wt-ppm, calculated as mass equivalents of acetone.

33. A plant for producing dimethyl ether by catalytic dehydration of crude methanol as feedstock in the gas phase, the plant comprising a reactor filled with dehydration catalyst and being configured so as to carry out a method of:
(a) providing crude methanol from methanol synthesis, the crude methanol having a total content of carbonyl compounds of not more than 100 wt-ppm, calculated as mass equivalents of acetone,
(b) evaporating the crude methanol and adjusting a reaction temperature and a reaction pressure,
(c) charging the reactor with the evaporated crude methanol with a defined space velocity,
(d) discharging a gaseous product mixture comprising dimethyl ether, non-reacted methanol and water, and
(e) cooling, partially condensing and separating the gaseous product mixture so as to provide gaseous dimethyl ether, liquid water and methanol as products, and recirculating the methanol product to step (a).
